(19) United States
(12) Patent Application Publication
(10) Pub. No.: US 2014/0161917 A1
(43) Pub. Date: Jun. 12, 2014

(54) Use of Nesprin-2 Expression Modulators and Compositions Thereof

(71) Applicant: Avon Products, Inc., Suffern, NY (US)

(72) Inventors: John W. Lyga, Basking Ridge, NJ (US); Ying Chen, Towaco, NJ (US); Qian Zheng, Morris Plains, NJ (US); Uma Santhanam, Tenafly, NJ (US)

(73) Assignee: Avon Products, Inc., Suffern, NY (US)

(21) Appl. No.: 14/066,862

(22) Filed: Oct. 30, 2013

Related U.S. Application Data
(60) Provisional application No. 61/735,645, filed on Dec. 11, 2012.

(51) Int. Cl. A61K 8/97; A61Q 19/00; A61Q 19/06; A61K 8/49; A61Q 19/08

(52) U.S. Cl. CPC A61K 8/97; A61K 8/494; A61Q 19/08; A61Q 19/06; A61Q 19/00
USPC 424/757; 424/725; 514/392; 435/6.13

(57) ABSTRACT

Methods of using nesprin-2 modulators to impart anti-aging benefits to the skin and/or improve skin conditions.

### CROSS-REFERENCE TO RELATED APPLICATIONS

This application claims priority to U.S. Provisional Patent Application Ser. No. 61/735,645, filed on Dec. 11, 2012. The entirety of each of the aforementioned applications is hereby incorporated by reference in its entirety for all purposes.

This application incorporates the entirety of the following documents by reference for all purposes:

U.S. patent application Ser. Nos. 13/305,779 and 13/216,626, filed on Dec. 30, 2010 and Aug. 24, 2011, respectively.

U.S. patent application Ser. No. 13/158,947, filed on Jun. 30, 2010, and U.S. patent application Ser. No. 13/324,150, filed on Dec. 13, 2011, the entirety of which is incorporated by reference for all purposes.

U.S. patent application Ser. No. 12/345,707, filed on Dec. 30, 2008; and Ser. No. 12/827,001, filed on Jun. 30, 2010.

U.S. patent application Ser. No. 12/747,364, filed on Dec. 21, 2007, granted U.S. Pat. No. 8,450,353 on May 28, 2013.

PCT Application Serial No. PCT/US12/68858, entitled “Serissa japonica extracts and methods of use”, filed on Dec. 11, 2012 and naming Qian Zheng as first inventor.

PCT Application Serial No. PCT/US12/68856, entitled “Callistephus chinensis extracts and methods of use”, filed on Dec. 11, 2012 and naming Qian Zheng as first inventor.

PCT Application Serial No. PCT/US12/68865, entitled “Hoya carnosa extracts and methods of use”, filed on Dec. 11, 2012 and naming Qian Zheng as first inventor.

### FIELD OF INVENTION

The present invention relates generally to compositions for topical application to the skin which comprise at least one Nesprin-2 modulator and the use of such compositions to provide benefits to the skin, in particular, improving the condition and appearance of skin affected by aging and/or photoaging.

### BACKGROUND OF THE INVENTION

There is active interest in the cosmetics industry in developing products that may be applied topically to the skin to counteract adverse changes in the skin. Cosmetic products that reverse or forestall such changes are increasingly in demand. Consumers continually seek to improve the appearance of their skin and in particular to reduce visible signs of skin aging. Unwanted signs include lines and wrinkles, skin sagging or atrophy, loss of suppleness, thickness, plumpness, tautness, elasticity, resiliency, and firmness, and there remains a need for products that combat such signs of aging and, more generally, that provide anti-aging and/or anti-wrinkle effects.

Over the years, a variety of approaches for treating these skin irregularities have been offered. Numerous dermatologic creams, lotions, vitamins, and herbal supplements have been proposed. Further, private spas and salons have offered massages, scrubs, wraps, compresses, essential oils, and herbal products to address the irregular skin contours. Most of these therapies do not provide a lasting remedy to these skin irregularities and require multiple treatments on an ongoing basis, often at considerable expense, to maintain any effect. Many of these treatments often pose the risk of serious side effects.

Nesprins are a family of nuclear membrane proteins characterized by spectrin repeats and a C-terminal transmembrane domain, called the KASH-domain, which anchors them in the nuclear membrane. Nesprins are components of the LING complex, linker of nucleoskeleton and cytoskeleton. On the nuclear side, they interact with SUN proteins, which are also anchored at the nuclear envelope. They also interact with lamin A/C, which forms the laminin network underneath the inner nuclear membrane and maintains nuclear architecture and integrity. In the cytoplasm, they interact with either actin cytoskeleton, or microtubules, or the intermediate filaments. See, e.g., Zhang et al., 2001 Journal of Cell Science 114, 4485-4498; Padmakumar et al., 2005 Journal of Cell Science 118, 3419-3430; Libotte et al., 2005 Molecular Biology of the Cell Vol. 16, 3411-3424, each of which is incorporated in its entirety for all purposes. Uncoupling of the nucleoskeleton and cytoskeleton due to either Nesprin1 or Nesprin-2 mutation can lead to Emery-Dreifuss muscular dystrophy (EDMD) in humans, which is a genetic neuromuscular disorder associated with skeletal muscle weakness and wasting, and cardiomyopath. See, e.g., Zhang et al., 2007. Human Molecular Genetics, Vol. 16, No. 23 2816-2833; Puckelwartz et al., 2009. Human Molecular Genetics, Vol. 18, No. 4 607-620, each of which is herein incorporated by reference in its entirety.

Nesprin-2, also termed nuclear envelope spectrin repeat protein 2; nucleus and actin connectine element protein; and/or synaptic nuclear envelope protein 2, is the predominant family member expressed in skin and the only nesprin family member that interacts with both the actin cytoskeleton and the microtubule network. Nesprin-2 affects cell polarity and alignment. In nesprin-2 knockout keratinocytes, nuclear shape is severely affected, leading to giant nuclei and nuclear envelop blebbing. In fibroblasts, loss of Nesprin-2 affects cell migration and cell polarity, resulting in reduced velocity and disoriented Golgi structure and MTOC (microtubule organizing center). Nesprin-2 also affects tissue regeneration in wound repair by regulating the cytoskeleton and affecting signaling processes. See, e.g., Luke et al., 2008. Journal of Cell Science 121, 1887-1898; Rashmi et al., 2012. Nucleus 3:2, 1-15, each of which is incorporated herein by reference for all purposes.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

The current invention relates to a method for improving the appearance of skin affected by a skin irregularity by topically applying thereto an effective amount of at least one modulator of nesprin-2 expression, in a cosmetically acceptable vehicle for a time sufficient to achieve an improvement in the appearance of said skin. In one embodiment, nesprin-2 expression is upregulated. In another embodiment, nesprin-2 expression is downregulated.

In one embodiment of the current invention, the modulator may be an extract of an extract of Justicia ventricosa, an extract of Tiliacora triandara, an extract of Ixora chinensis, an extract of Operculina turpethum, D-desthiobiotin or a derivative thereof, an extract of Archidendron clyperia, an extract of Medemia nobilis, or a combination thereof. In another embodiment, the modulator may be an extract of Serrisa japonica, an extract of Callistephus chinensis, an extract of Hoya carnosa, or a combination thereof. In another embodiment, at least two modulators are applied. In another embodiment, the modulator is not an extract of Tiliacora triandra, Medemia nobilis, Ixora chinesis, Operculina turpethum, Justicia ventricosa, Archidendron clyperia, and/or is not D-desthiobiotin.

Another embodiment is directed to the current method wherein the composition is applied at least once daily for a period of time sufficient to improve the appearance of the skin. In accordance with the current invention the composition may be a leave on composition. In certain embodiments of the current invention, the effective amount of the modulator is about 0.001% to about 25% by weight, in one embodiment about 0.001% to about 1% by weight. Further, an embodiment of the current invention is directed to the use of the method of the current invention daily for a period of four weeks.

A further aspect of the current invention is directed to a method for screening active agents useful for improving the aesthetic appearance of skin that involves assaying candidate substances for their ability to modify nesprin-2 expression. In a further embodiment of this screening method, the assaying step involves incubating dermal fibroblasts with said candidate substance and subsequently measuring the levels of mRNA encoding nesprin-2 expression. The step of measuring may be carried out by quantitative polymerase chain reaction (qPCR) in other embodiments of the current invention.

The current embodiment is further directed to a method of treating the skin comprising topically applying to an area of the skin in need thereof an effective amount of an active agent that modulates nesprin-2 expression, wherein the ability of said active agent to modulate nesprin-2 expression has been determined by an assay which measures the level of mRNA encoding nesprin-2 in a cell that has been contacted with said active agent.

These and other aspects of the present invention will be better understood by reference to the following detailed description and accompanying figures.

### DESCRIPTION OF THE DRAWINGS

FIG. 1 depicts nesprin-2 gene expression in neonatal and aged dermal fibroblasts.

FIG. 2 is an HPLC profile of an extract of S. japonica

FIG. 3 is an HPLC profile of an extract of C. chinensis

FIG. 4 is an HPLC profile of an extract of H. carnosa

FIG. 5 is an HPLC profile of an extract of Tiliacora triandra

FIG. 6 is an HPLC profile of an extract of M. nobilis

FIG. 7 is an HPLC profile of an extract of I. chinensis

FIG. 8 is an HPLC profile of an extract of J. ventricosa

FIG. 9 is an HPLC profile of an extract of A. clyperia

FIG. 10 is an HPLC profile of an extract of O. turpethum

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, and some features may be exaggerated to show details of one embodiment components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

The current invention provides for a new and novel method of treating, preventing, and/or forestalling various skin irregularities through the administration of nesprin-2 modulators to skin in need thereof. In particular, the nesprin-2 modulators seek to treat, prevent, or forestall skin irregularities, such as impaired cytoskeleton and/or nuclear envelope integrity, loss of proper cell polarity and alignment, and/or disregulation of wound healing and skin regeneration, by modulating nesprin-2 expression and rejuvenating skin affected by impaired cytoskeleton and/or nuclear envelope integrity, loss of proper cell polarity and/or alignment, and disregulation of wound healing and/or skin regeneration.

One aspect of the present invention relates to compositions for topical application which comprises an effective amount of a nesprin-2 modulator to treat, reverse, ameliorate and/or prevent various conditions characterized by suboptimal expression of nesprin-2. Such benefits include without limitation, the following:

(a) reduction in the effects of impaired cytoskeleton and/or nuclear envelope integrity, loss of proper cell polarity and alignment, and/or disregulation of wound healing and skin regeneration.impaired cytoskeleton and/or nuclear envelope integrity, loss of proper cell polarity and/or alignment, and disregulation of wound healing and/or skin regeneration,

(b) reduction in pitting appearance of skin resulting from impaired cytoskeleton and/or nuclear envelope integrity, loss of proper cell polarity and alignment, and/or disregulation of wound healing and skin regeneration.impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging upon squeezing;

(c) reduction in the extent of area affected by impaired cytoskeleton and/or nuclear envelope integrity, loss of proper cell polarity and alignment, and/or disregulation of wound healing and skin regeneration impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging;

(d) prevention or delay in the recurrence of impaired cytoskeleton and/or nuclear envelope integrity, loss of proper cell polarity and alignment, and/or disregulation of wound healing and skin regeneration which may lead to lines/wrinkles, sagging, and other signs of aging and/or photoaging;

(e) improvement in collagen disposition;

(f) improvement in adipocyte/fat tissue disposition;

(g) reduction in sagging of the skin; and

(h) reduction in the extent of the area affected by sagging skin.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, sec. 201(i).

In practice, the compositions of the invention are applied to skin in need of treatment, i.e., skin which suffers from a deficiency or loss in any of the foregoing attributes or which would otherwise benefit from improvement in any of the foregoing skin attributes. In certain preferred embodiments the compositions and methods of the invention are directed to the prevention, treatment, and/or amelioration of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging. In this case, the compositions are applied to skin in need of treatment, by which is meant skin exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging normally or upon squeezing. In one embodiment, the compositions are applied directly to the area of the skin exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging. The compositions and methods are suitable for treating impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging on any surface of the skin, including without limitation, the buttocks, thighs, hips, or limbs. Additionally, the compositions of the current invention can be used to treat, ameliorate, and/or prevent/delay the appearance of sagging facial or neck skin, in particular periorbital bulging, and are applied to the sagging area in need of treatment.

All terms used herein are intended to have their ordinary meaning unless otherwise provided.

As used herein, the term “consisting essentially of” is intended to limit the invention to the specified materials or steps and those that do not materially affect the basic and novel characteristics of the claimed invention, as understood from a reading of this specification. All percentages are by weight based on the total weight of the composition, unless otherwise indicated.

By “cosmetically acceptable” it is meant that a particular component is generally regarded as safe and nontoxic at the levels employed.

The term “active amount” refers to the amount of nesprin-2 modulator, absent diluent, solvent, carrier, filler or any other ingredient. An “amount effective” or an “effective amount” to provide a particular anti-aging benefit to the skin refers to the “active amount” of extract required to provide a clinically measurable improvement in the particular manifestation of skin aging when applied for a time sufficient to provide a clinically measurable improvement in the particular manifestation of downregulated nesprin-2.

The phrase “individual in need thereof” refers to a human who could benefit from improved dermal appearance or health, including males or females.

As used herein, the terms “prevent,” “preventing,” etc. mean delaying the onset of, hindering the progress of, hindering the appearance of, protection against, inhibiting or eliminating the emergence of, or reducing the incidence of various cosmetic or dermatologic conditions, damages, effects or symptoms. Use of the term “prevention” is not meant to imply that all subjects in a subject population administered the cosmetic composition will always be unaffected by or fail to develop the cosmetic or dermatologic conditions, damage, effect or symptom, but rather that the subject population will exhibit a reduction in the cosmetic or dermatologic damages, effects, or symptoms. For example, many flu vaccines are not 100% effective in preventing the flu in those administered the vaccine.

The term “modulator” encompasses any substance, including, without limitation, organic molecules; biomolecules (e.g., peptides, proteins, antibodies, nucleic acid oligomers, etc.); and combinations of substances, such as botanical extracts. The modulators regulate the cellular levels of at least one isoform of nesprin-2, by which is meant that the cellular levels of nesprin-2 are either increased or decreased by the active agent. The term “modulation” may refer to up-regulation, induction, stimulation, potentiation, and/or relief of inhibition, as well as inhibition, attenuation and/or downregulation or suppression. The modulators may be, without limitation, activators or agonists, which are compounds that, for example, bind to, stimulate, increase, open, activate, facilitate, enhance activation, sensitize, or up-regulate expression levels of genes or nesprin-2 or peptides. The modulators may also be, without limitation, inhibitors or antagonists, which are, for example, compounds that bind to, partially or totally block stimulation, decrease, prevent, delay activation, inactivate, desensitize, or downregulate expression levels of genes or nesprin-2. The mechanism by which the protein level is modulated is not important.

As used herein, the term “expression levels” refers to an amount of a gene and/or protein that is expressed in a cell. As used herein, a “gene” includes a polynucleotide containing at least one open reading frame that is capable of encoding a particular polypeptide. As used herein, the terms “polynucleotide” is synonymous with “oligonucleotide” and includes polymeric forms of nucleotides of any length, either deoxyribonucleotides or ribonucleotides, or analogs thereof, including, without limitation, mRNA, DNA, cDNA, primers, probes, and the like.

In one embodiment, an assay is provided for determining the expression levels of nesprin-2 after a cell has been treated, incubated, or otherwise contacted with a candidate substance. The term “candidate substance” refers to any substance that is tested for activity as a modulator of nesprin-2, whether or not the substance is suspected of possessing such activity. The cell can be any cell that expresses nesprin-2. In one embodiment, the cell is a dermal fibroblast or precursor thereof. In another embodiment, the cell is a human or mouse cell. After the cell has been incubated with a candidate substance for a sufficient length of time to provide a measurable change in expression levels, which will typically be at least one hour, and more typically from about 72 hours to 144 hours (3 to 6 days) it is then lysed to release the cellular components, such as nesprin-2 mRNA encoding those proteins. The amount of nesprin-2-encoding mRNA, cDNA or any other resultant substance indicating relative nesprin-2 expression may then be measured by any suitable technique for detection and quantitation of peptides and proteins and/or polynucleotides (e.g., mRNA).

One method for measuring expression levels of nesprin-2 protein involves the quantitation of mRNA expression. Suitable methods for determining mRNA expression include quantitative PCR (QPCR), real-time QPCR, reverse transcription PCR (RT-PCR), and quantitative reverse transcription PCR (QRT-PCR), as are well-known in the art. As described in detail in U.S. Pat. Nos. 7,101,663 and 7,662,561, the disclosures of which are hereby incorporated by reference, a quantitative reverse transcriptase polymerase chain reaction (QRT-PCR) for detecting mRNA may include the steps of: (a) incubating an RNA sample from the cellular lysate with a reverse transcriptase and a high concentration of a target sequence-specific reverse transcriptase primer under conditions suitable to generate cDNA; (b) subsequently adding suitable polymerase chain reaction (PCR) reagents to the reverse transcriptase reaction, including a high concentration of a PCR primer set specific to the cDNA and a thermostable DNA polymerase to the reverse transcriptase reaction; and (c) cycling the PCR reaction for a desired number of cycles and under suitable conditions to generate PCR products (“amplicons”) specific to the cDNA. The products of the QRT-PCR process may be compared after a fixed number of PCR cycles to determine the relative quantity of the RNA species as compared to a given reporter gene, for example, by Southern blotting. More typically, the progress of the PCR reaction is monitored by analyzing the relative rates of amplicon production for each PCR primer set, for example, by (1) non-specific fluorescent dyes that intercalate with any double-stranded DNA, and/or (2) sequence-specific DNA probes consisting of oligonucleotides that are labeled with a fluorescent reporter which permits detection only after hybridization of the probe with its complementary DNA target. The mRNA may be any mRNA known to one of ordinary skill of the art that is associated with the protein of interest (in one embodiment, nesprin-2).

The level of expression in the above disclosed methods of determining nesprin-2 expression levels may be compared to controls that are not treated with the candidate substance to determine the relative degree of modulation. In some embodiments, the candidate substance will up-regulate mRNA expression by at least about 10%, more suitably at least about 20%, at least about 30%, at least about 40%, or at least about 50%. In one embodiment, the candidate substance will up-regulate mRNA expression by at least about 60%, at least about 70%, at least about 80%, at least about 90%, or at least about 100%. Candidate substances meeting these criteria may be selected for use of for further evaluation.

Nesprin-2 modulators, identified in accordance with the procedures noted above as shown by Example 2 below, can include naturally occurring or synthetic peptides, amino acids, and chemicals entities such as, but not limited to, a 1-[(1H-pyrazol-4-yl)carbonyl]heterocyclic compound and/or D-desthiobiotin. Further, nesprin-2 modulators may include botanical extracts such as extracts of: Justicia ventricosa, Tiliacora triandara, Ixora chinensis, Operculina turpethum, D-desthiobiotin or a derivative thereof, Archidendron clyperia, Medemia nobilis, Serrisa japonica, Callistephus chinensis, Hoya carnosa, or a combination thereof.

Medemia nobilis is a dioecious palm that is native to Madagascar. It produces inflorescences, about 1 m long, with thick, catkin-like branches covered with inconspicuous flowers, and is described, for example, in U.S. patent application Ser. Nos. 13/305,779 and 13/216,626, filed on Dec. 30, 2010 and Aug. 24, 2011, respectively, the entirety of which are incorporated by reference for all purposes.

Ixora chinesis is a flowering plant native to southern China that is characterized by its almost stalkless leaves and red flowers. It is used to treat various ailments like rheumatism and wounds, and is described, for example, in U.S. patent application Ser. No. 13/158,947, filed on Jun. 30, 2010, the entirety of which is incorporated by reference for all purposes.

Operculina turpethum (“Trivit”) is a large stout perennial twinner that has milky juice and fleshy roots. It is thought to have activity against liver disorders, and also has anthelmintic, expectorant, antipyretic, anti-inflammatory, and purgative properties, and is described, for example, in U.S. patent application Ser. No. 13/158,947, filed on Jun. 30, 2010, the entirety of which is incorporated by reference for all purposes.

Tiliacora triandra (“Yanang”) is a species of flowering plant native to mainland Southeast Asia and used particularly in the cuisines of northeast Thailand and Laos. In traditional Southeast Asian medicine, Tiliacora triandra has been used as an herbal medicine for fever relief, alcohol intoxication, inflammation, and bacterial/fungal infection. For instance, the use of Tiliacora triandra Diels against plasmodium falciparum (cause malaria in humans) is disclosed in Pavanand et al., Phytother. Res., 3, 215-217 (1989), and is described, for example, in U.S. patent application Ser. No. 12/345,707, filed on Dec. 30, 2008; Ser. No. 13/158,947, filed on Jun. 30, 2010; and Ser. No. 12/827,001, filed on Jun. 30, 2010, the entirety of which is incorporated by reference for all purposes.

Justicia ventricosa is a species of the acanthaceae family, native to China, Burma (Myanmar), India, and Pakistan. It has medium-sized leaves and it produces small white flowers with red spots. It has been used in traditional Chinese medicine, and it is believed to invigorate the circulation of blood, remove hemorrhage, congestion, thrombosis, and local ischemia (microclots) and tissue changes, alleviate lower back pain and pain in lower extremities, traumatic injury, sprain, and arthritis, and is described, for example, in U.S. patent application Ser. No. 13/216,626, filed on Aug. 24, 2011, the entirety of which is herein incorporated by reference for all purposes.

Archidendron clyperia is a small evergreen tree, in the fabaceae family. It typically has small leaves and produces small while-yellow flowers and orange-red fruits coiled with black seeds. Its leaves have been used for tanning and coloring rattan. It is found in India, Burma (Myanmar), southern China, Malaysia, Thailand, Sri Lanka, Laos, and also in Borneo. It is also known as Inga clypearia, Jack Pithecellobium subcoriaceum, and in Tamil as Malai-vagai, Mazhavagai. In Borneo it is called Anup-anup, Jerung, Kangkat rangkat, Kelayung, Petai kerayung, and Tambilit, and is described, for example, in U.S. patent application Ser. No. 13/216,626, filed on Aug. 24, 2011, the entirety of which is herein incorporated by reference for all purposes.

D-desthiobiotin is generally described in U.S. patent application Ser. No. 12/747,364, filed with a priority date of Dec. 21, 2007, herein incorporated by reference in its entirety for all purposes.

Serrisa japonica, also known as the tree of a thousand stars, is an evergreen shrub with small, bright green, oval leaves that is native to open sub-tropical woodlands and wet meadows in southeast Asia. Depending on the variety, small white or pink flowers cover the tree in late spring. The natural color of the trunk is gray and the bark roughens with age.

China aster, also called Annual Aster, (Callistephus chinensis), is a herbaceous plant of the aster family. Many cultivated varieties of Callistephus chinensis exist and are longtime garden favorites. The species originated in China and is typically about 2.5 feet tall with white to violet flowers with yellow centers. The use of Callistephus chinensis has been reported as an ingredient in an elixir to treat diabetes, see JP 1056619, and as a component in a facial mask used to treat acne and dermatitis, see KR2000049439.

Hoya carnosa, the wax plant, is a species in the dogbane family (Apocynaceae). It is native to Eastern Asia and Australia. Hoya carnosa has star-shaped light pink flowers covered in tiny hairs that are borne in clusters. They are heavily scented.

An example of a 1-[(1H-pyrazol-4-yl)carbonyl]heterocyclic compound is generally described, for example, in French Patent Application FR2924931A1, herein incorporated by reference in its entirety for all purposes.

The above-noted extracts contain a number of active compounds, one or more of which modulate nesprin-2.

The plant materials may be in any form including, but not limited to, the whole plant, a dried plant, a ground plant, or parts thereof, including but not limited to, seeds, needles, leaves, roots, bark, cones, stems, rhizomes, callus cells, protoplasts, flowers, and meristems, or components and/or constituents found in, or isolated from, the natural plant material, and/or portions of the plant, or any combinations thereof. In one embodiment, the natural plant material is in the form of an extract derived from the whole plant or from a select portion of the plant, such as the leaves of the plant. It is to be understood that “natural plant material” also includes an ingredient, component, constituent, or extract derived from the natural plant material.

Specifically, the botanical component is derived from raw materials collected from the plants, which may contain the desired constituent(s), such as seeds, needles, leaves, roots, bark, cones, stems, rhizomes, callus cells, protoplasts, organs and organ systems, and meristems. In certain embodiments, the raw materials collected from the plants are ground to small particle sizes. In addition, the raw materials may be dried to reduce water content. The raw materials may be dried by a number of different means, such as, for example, air-dried, oven-dried, rotary evaporated under vacuum or lyophilized.

The extract of the above-noted plants may be obtained by distilling the raw materials with a stripping agent. The stripping agent may be a liquid that is miscible, immiscible, or partially miscible with the desired extract from the plants. Suitable stripping agents include, but are not limited to, water; alcohols (such as methanol, ethanol, propanol, butanol and the like); glycols; ethers (such as diethyl ether, dipropyl ether, and the like); esters (such as butyl acetate, ethyl acetate, and the like); ketones (such as acetone, ethyl methyl ketone, and the like); dimethyl sulfoxide; acetonitrile; other organic solvents; and combinations thereof. In one embodiment, the stripping agent is immiscible with the desired extract (e.g., essential oil) from the plant. In one embodiment, the stripping agent is water. More In one embodiment, the extract is obtained by steam distillation. The extract (e.g., essential oil) may be collected by phase separation from the stripping agent. It is believed that the stripping agent increases the overall vapor pressure of a distillation system for obtaining an extract and thereby reducing the boiling point of the desired product, the extract.

In other embodiments, the botanical component may be in the form of an extract obtained by solvent extraction, in one embodiment obtained by an organic solvent extraction. Briefly, the organic solvent extraction method involves washing and extracting the raw materials, which may be whole or ground into small particle sizes, using an organic solvent. Non-limiting examples of organic solvents include methanol, ethanol, isopropanol, dichloromethane, chloroform, hexane, xylene, and petroleum ether. An extracting machine may be used for organic solvent extraction as is well known in the field. The raw materials are pushed in the extracting machine by a thruster, which slowly moves the plant raw materials forward. Organic solvent (e.g., ethanol) may be added into the machine through a solvent inlet at the top of a waste discharge outlet. Due to the difference in gravity and equilibrium, the solvent flows toward the raw material inlet, soaks the materials and flows out from the opposite side of the solvent inlet. Since the plant materials and the solvent move in opposite directions against each other, the plant materials are constantly immersed in a solution that contains a low-concentration of extract. As a result of equilibrium, high yield of plant constituent(s) may be achieved by continuously extracting the plant material against the low-concentration solution.

An extraction time suitable to extract the plant constituents is used, typically between about 1-10 hours is suitable, and more In one embodiment is between about 2-8 hours, and most In one embodiment is between about 3-6 hours. The temperature of extraction is between about 30° C.-100° C., In one embodiment between about 40° C.-70° C., and more In one embodiment between about 50° C.-60° C. The collected extract is then fine-filtered to remove debris, and may be used directly, or is concentrated, for example by distilling the solvent or by other conventional processing. The solution of extract actives may be rotary evaporated under vacuum or lyophilized. A typical extract's actives content is above about 25%, In one embodiment above 50%, and the extract can also be provided an essential oil or a concentrate having a semi-solid or solid consistency.

Similarly, aqueous-organic solvent extraction involves initially collecting raw materials from the above-noted plants, which may be whole or ground into small particle sizes. The ground plant material is soaked in aqueous solution that is acidic or alkaline, depending on the solubility and stability of the desired extract under acidic or alkaline (basic) conditions. For extraction under acidic conditions, an acid such as hydrochloric acid or sulfuric acid is added to water, e.g., at a concentration of about 3% (w/v). For extraction under alkaline conditions, an alkali such as sodium hydroxide or sodium carbonate is added to water. The extraction time and temperature of extraction are typically similar to that used in the organic solvent extraction method described above.

The extract is then collected and fine-filtered to remove debris. Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, concentrated or dried. Alternatively, organic solvent may then be added to the neutralized solution to transfer the extract from an aqueous phase to an organic phase. Examples of such organic solvents include, but are not limited to, ethanol, isopropanol, butanol, pentanol, hexanol and xylene. The extract comprising the transferred extract actives dissolved in organic solvent may be used directly as an essential oil or a concentrate, or dried by a number of different means, such as, for example, air-dried, oven-dried, rotary evaporated under vacuum or lyophilized to a semi-solid or solid consistency.

It should also be noted that different plants containing different constituents can be mixed and extracted together. This process of mixed extraction can In one embodiment be used for extracting those plants containing constituents with similar solubility in the solvent used for extraction, such as ethanol. The mixture of extracts can be concentrated and stored in an appropriate solvent.

Examples of extraction of Justicia ventricosa, Tiliacora triandara, Ixora chinensis, Operculina turpethum, Archidendron clyperia, Medemia nobilis, Serrisa japonica, Callistephus chinensis, Hoya carnosa, Justicia ventricosa, Tiliacora triandra, Ixora chininensis, Operculina turpethum, Archidendron clyperia, and/or Medemia nobilis, may be provided below and/or in the incorporations by reference described above.

In another embodiment, extract as used herein, also includes “synthetic” extracts, i.e., various combinations of known plant components and/or constituents that are combined to substantially mimic the composition and/or activity of any one or more of the above-noted plant extracts of natural origin having nesprin-2 modulating activities. In one embodiment, the synthetic extracts have substantially the same number of active components as the natural plant material. The correspondence of the numerical incidence of actives between the synthetic extracts and the natural plant material may also be described in terms of “percent commonality.” The synthetic extract has about 50 percent or more commonality to the chemical composition of a plant or natural extract. In other words, the synthetic extract has about 50 percent or more of the active ingredients found in the plant or a natural extract. More In one embodiment, the chemical composition of the synthetic extract has about 70 percent or more commonality to the chemical composition of a plant or a natural extract. Optimally, a synthetic extract has about 90 percent or more commonality to the chemical composition of a plant or a natural extract.

The cosmetic compositions according to the invention can be formulated in a variety of forms for topical application and will comprise from about 0.00001% to about 90% by weight of one or more actives that modulate nesprin-2 proteins, in one embodiment comprising such actives in an amount from about 0.001% to about 25% by weight, and in another embodiment from about 0.001% to about 1% by weight.

Another embodiment of the invention encompasses compositions comprising a cosmetically or dermatologically acceptable formulation which is suitable for contact with living animal tissue, including human tissue, with virtually no adverse physiological effect to the user. Compositions embraced by this invention can be provided in any cosmetically and/or dermatologically suitable form, in one embodiment as a lotion or cream, but also in an anhydrous or aqueous base, as well as in a sprayable liquid form. Other suitable cosmetic product forms for the compositions of this invention include, for example, an emulsion, a cream, a balm, a gloss, a lotion, a mask, a serum, a toner, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as colorants, fragrances, emollients, humectants, preservatives, vitamins, chelators, thickeners, perilla oil or perilla seed oil (WO 01/66067 to a “Method of Treating a Skin Condition,” incorporated herewith) and the like, as well as other botanicals such as aloe, chamomile, and the like, and as further described below.

Also, embraced by the invention are transdermal modes of delivery, such as patches and the like, with or without suitable penetration enhancers. The methods and compositions embodied by the invention provide a means by which the nesprin-2 modulators can be effectively administered in a transdermal system. Accordingly, a transdermal means of delivering a composition or formulation (often with a penetration enhancing composition) to the skin is that of the transdermal patch or a similar device as known and described in the art. Transdermal patches are designed to deliver an effective amount of compound across a user's skin. Transdermal patches typically involve a liquid, gel, solid matrix, or pressure-sensitive adhesive carrier into which the nesprin-2 modulator may be incorporated. Patch formulations and preparation are well known in the art. See for example “Dermatological and Transdermal Formulations” (Drugs and the Pharmaceutical Sciences, Vol 119) by Kenneth A Walters (Editor), Marcel Dekker and “Transdermal Drug Delivery” (Drugs & the Pharmaceutical Sciences) by Richard H. Guy (Editor), Jonathan Hadgraft (Editor) 2nd Rev& ex edition Marcel Dekker and “Mechanisms of Transdermal Drug Delivery” (Drugs & the Pharmaceutical Sciences, Vol 83) edited by Russell O. Potts and Richard H. Guy (1997). Examples of such devices are disclosed in U.S. Pat. Nos. 5,146,846; 5,223,262; 4,820,724; 4,379,454; and 4,956,171; and U.S. Patent Publication No. US20110300198, all of which are incorporated herein by reference and such descriptions are not meant to be limiting. The transdermal mode of storing and delivering the compositions onto the skin, including hair, and forming the active composition is convenient and well-suited for the purposes of an embodiment of the present invention. In a preferred method, the application is through a sustained release vehicle, carrier, or diluent, e.g., a topically applied sustained released patch. In one embodiment, when a topical patch is used, the patch is applied to the desired area for extended period of time. In one embodiment, the extended period of time is greater than one hour, in one embodiment the extended period of time is overnight, i.e., when the user is sleeping. In a further embodiment of the current invention, the transdermal patch may be applied to skin exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging or at risk for exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging, i.e., the buttocks, thighs, hips, or limbs for extended periods of time, at least one day, two or more days, at least a week, or longer if necessary in order to provide prolonged exposure to the nesprin-2 modulators in order to achieve the desired enhancements of the skin in need of treatment.

The compositions can include a cosmetically acceptable vehicle. Such vehicles may take the form of any known in the art suitable for application to skin and may include water (e.g., deionized water); vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, in one embodiment organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures of the foregoing.

The vehicle may comprise an aqueous phase, an oil phase, an alcohol, a silicone phase or mixtures thereof. The cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant.

The oil phase of the emulsion, in one embodiment, has one or more organic compounds, including emollients; humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

The compounds suitable for use in the oil phase include without limitation, vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; and the like. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils include those having 6-20 carbon atoms may be utilized, in one embodiment having 10-16 carbon atoms. Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99A™ are also contemplated to be suitable. Various commercially available C₁₆ isoparaffins, such as isohexadecane (having the tradename Permethyl®) are also suitable. Examples of preferred volatile hydrocarbons include polydecanes such as isododecane and isodecane, including for example, Permethyl-99A (Presperse Inc.) and the C₇-C₈ through C₁₂-C₁₅ isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, polyethylene waxes, Fischer-Tropsch waxes, beeswax, microcrystalline wax, silicone waxes, fluorinated waxes, and any combination thereof.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. The preferred emulsifiers include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11^{th} Edition 2006, the disclosure of which is hereby incorporated by reference.

These emulsifiers typically will be present in the composition in an amount from about 0.001% to about 10% by weight, in particular in an amount from about 0.01% to about 5% by weight, and in one embodiment, from about 0.1% to about 3% by weight.

The oil phase may comprise one or more volatile and/or non-volatile silicone oils. Volatile silicones include cyclic and linear volatile dimethylsiloxane silicones. In one embodiment, the volatile silicones may include cyclodimethicones, including tetramer (D₄), pentamer (D₅), and hexamer (D₆) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics”, Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are preferred non-volatile silicone oils. The non-volatile silicone oils will typically have a viscosity from about 10 to about 60,000 centistokes at 25° C., in one embodiment between about 10 and about 10,000 centistokes, and more preferred still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted will various functional groups such as alkyl, aryl, amine groups, vinyl, hydroxyl, haloalkyl groups, alkylaryl groups, and acrylate groups, to name a few.

The water-in-silicone emulsion may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference in its entirety. These emulsifiers generally comprise a polydiorganosiloxane backbone, typically polydimethylsiloxane, having side chains comprising -(EO)ₘ— and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. Preferred examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in particular in an amount from about 0.01% to about 5% by weight, in one embodiment, below 1% by weight.

The aqueous phase of the emulsion may include one or more additional solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like.

The oil-containing phase will typically comprise from about 10% to about 99%, in one embodiment from about 20% to about 85%, and in one embodiment from about 30% to about 70% by weight, based on the total weight of the emulsion, and the aqueous phase will typically comprise from about 1% to about 90%, in one embodiment from about 5% to about 70%, in one embodiment from about 20% to about 60% by weight of the total emulsion.

The compositions may include liposomes. The liposomes may comprise other additives or substances and/or may be modified to more specifically reach or remain at a site following administration.

The composition may optionally comprise other cosmetic actives and excipients, obvious to those skilled in the art including, but not limited to, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, depigmenting agents, hypopigmenting agents, preservatives (e.g., DMDM Hydantoin/Iodopropynylbutylcarbonate), stabilizers, pharmaceutical agents, photostabilizing agents, neutralizers (e.g., triethanolamine) and mixtures thereof. In addition to the foregoing, the cosmetic compositions of the invention may contain any other compound for the treatment of skin disorders.

Colorants may include, for example, organic and inorganic pigments and pearlescent agents. Suitable inorganic pigments include, but are not limited to, titanium oxide, zirconium oxide and cerium oxide, as well as zinc oxide, iron oxide, chromium oxide and ferric blue. Suitable organic pigments include barium, strontium, calcium, and aluminium lakes and carbon black. Suitable pearlescent agents include mica coated with titanium oxide, with iron oxide, or with natural pigment.

Various fillers and additional components may be added. Fillers are normally present in an amount of about 0 weight % to about 20 weight %, based on the total weight of the composition, in one embodiment about 0.1 weight % to about 10 weight %. Suitable fillers include without limitation silica, treated silica, talc, zinc stearate, mica, kaolin, Nylon powders such as Orgasol™, polyethylene powder, Teflon™, starch, boron nitride, copolymer microspheres such as Expancel™ (Nobel Industries), Polytrap™ (Dow Corning) and silicone resin microbeads (Tospearl™ from Toshiba), and the like.

In one embodiment of the invention, the compositions may include additional skin actives such as, but not limited to, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, salicylic acid or salicylates, thiodipropionic acid or esters thereof, and advanced glycation end-product (AGE) inhibitors.

In a specific embodiment, the composition may comprise at least one additional botanical, such as, for example, a botanical extract, an essential oil, or the plant itself. Suitable botanicals include, without limitation, extracts from Abies pindrow, Acacia catechu, Anogeissus latifolia, Asmunda japonica, Azadirachta indica, Butea frondosa, Butea monosperma, Cedrus deodara, Emblica officinalis, Ficus benghalensis, Glycyrrhiza glabra, Ilex purpurea Hassk, Inula racemosa, Ligusticum chuangxiong, Ligusticum lucidum, Mallotus philippinensis, Mimusops elengi, Morinda citrifolia, Moringa oleifera, Naringi crenulata, Nerium indicum, Psoralea corylifolia, Stenoloma chusana, Terminalia bellerica, tomato glycolipid and mixtures thereof.

The composition may comprise additional active ingredients having anti-aging benefits, as it is contemplated that synergistic improvements may be obtained with such combinations. Exemplary anti-aging components include, without limitation, botanicals (e.g., Butea frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few.

Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In another embodiment, the topical compositions of the present invention may also include one or more of the following: a skin penetration enhancer, an emollient, a skin plumper, an optical diffuser, a sunscreen, an exfoliating agent, and an antioxidant.

An emollient provides the functional benefits of enhancing skin smoothness and reducing the appearance of fine lines and coarse wrinkles. Examples include isopropyl myristate, petrolatum, isopropyl lanolate, silicones (e.g., methicone, dimethicone), oils, mineral oils, fatty acid esters, cetyl ethylhexanoate, C12-15 alkyl benzoate, isopropyl isostearate, diisopropyl dimer dillinoeate, or any mixtures thereof. The emollient may be, in one embodiment, present from about 0.1 wt % to about 50 wt % of the total weight of the composition.

A skin plumper serves as a collagen enhancer to the skin. An example of a suitable, and preferred, skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

An optical diffuser is a particle that changes the surface optometrics of skin, resulting in a visual blurring and softening of, for example, lines and wrinkles. Examples of optical diffusers that can be used in the present invention include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. When present, the optical diffuser may be present from about 0.01 wt % to about 20 wt % of the total weight of the composition.

A sunscreen for protecting the skin from damaging ultraviolet rays may also be included. Preferred sunscreens are those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. A preferred exfoliating agent is glycolic acid. When present, the exfoliating agent may comprise from about 0.1 wt % to about 80 wt % of the composition.

Antioxidants scavenge free radicals from skin, protecting the skin from environmental aggressors. Examples of antioxidants that may be used in the present compositions include compounds having phenolic hydroxy functions, such as ascorbic acid and its derivatives/esters; alpha-hydroxyacids; beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives (e.g., tocopheryl acetate); uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may comprise an antioxidant, inone embodiment from about 0.001 wt % to about 10 wt %, and inone embodiment from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents such as bentonite, smectite, magnesium aluminum silicate and lithium magnesium silicate; metal chelating agents such as EDTA; pigments such as zinc oxide and titanium dioxide; colorants; emollients; and humectants.

In one embodiment, the composition of the invention comprising an nesprin-2 modulator may have a pH between about 1 and about 8. In certain embodiments, the pH of the composition will be acidic, i.e., less than 7.0, and in one embodiment will be between about 2 and about 7, inone embodiment between about 3.5 and about 5.5.

The invention provides a method for treating skin irregularities of through the topical application of a composition comprising an nesprin-2 modulator, in one embodiment in a cosmetically acceptable vehicle, over the affected area for a period of time sufficient to reduce, ameliorate, reverse or prevent the skin irregularity. This method is particularly useful for treating impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging.

Cosmetic compositions taught herein can be applied to an area of skin affected by impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging to improve the appearance of the skin. An improvement may involve a improvement of/reversal of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging In certain embodiments, known evaluative scales can be used to determine the initial severity of the impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging and gauge the improvement after treatment with the cosmetic compound of the current invention.

In some embodiments, a method is provided for reducing the re-occurrence of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging. Reducing the re-occurrence refers to delaying the recurrence of any impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging on a previously-affected area, or reducing the extent of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging that re-appears on the area, such that any recurrent impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging is less visible than previous amounts.

Similarly, compositions comprised of the nesprin-2 modulators of the current invention may be applied to sagging areas of the facial or neck skin such as around the eyes, cheeks, jowls, forehead, and neck in order to obtain an improvement in the appearance of the skin, i.e., a reduction in the severity of sagging, a reduction in the puffiness of the sagging, a reduction in the size of the area affected by sagging. Further, the compositions of the current invention may be applied to the area of skin at risk of sagging in an effort to prevent, forestall, or delay the appearance of such sags. In a preferred embodiment, the method of the current invention is used to address periorbital bulging, i.e. a sagging below and/or above the eye.

Exemplary actives used to prevent, treat, reverse, and/or ameliorate impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging agents include, without limitation, phosphodiesterase inhibitors, such as xanthine analogs, caffeine, aminophylline, and theophylline; adenylate cyclase activators, such as forskolin and Coleus forskohlii extract; lipolysis stimulators, such as hawthorne extract and cola extract; beta adrenergic receptor agonists, such as isoproterenol; alpha-2-adrenergic antagonists, such as yohimbine and Ginkgo biloba extract; perilla oil (see, e.g., U.S. Pat. No. 7,410,658, incorporated herein by reference in its entirety); carnitine and/or creatine (see, e.g., US 2007/0264205 entitled “Cosmetic Composition having Carnitine Creatinate and Methods for Using,” incorporated herein by reference in its entirety). In some embodiments, additional actives may include a collagen stimulator and/or an elastin stimulator, e.g., a substance that stimulates elastin production, and/or a glycosaminoglycan enhancer. Examples of collagen, elastin and glycosaminoglycan enhancers include, e.g., fennel extract, carrot extract, and alfalfa extract. In some embodiments, the additional actives may include a collagenase inhibitor and/or elastase inhibitor. In some embodiments, the invention relates to synergistic action of one or more compositions described herein with perilla oil, e.g., to provide enhanced effects to reverse, prevent and/or minimize impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging benefits to skin.

In some embodiments, the cosmetic compositions can further comprise at least one collagen and/or elastin stimulator. Such collagen or elastin stimulators are effective in, for example, providing improvement in procollagen and/or collagen production and/or improvement in maintenance and remodeling of elastin.

Accordingly, the invention provides novel mechanisms of action to improve the appearance of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging, and thus provides for potent methods of preventing, treating and/or minimizing impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging compositions for use thereon.

Without wishing to be bound by any particular theory, it is believed that the compositions of the present invention enhance and improve the aesthetic appearance of skin by improving/strengthening the nucleoskeleton/cytoskeleton coupling at least partially facilitated by nesprin-2.

In a further embodiment of the current invention, the nesprin-2 modulators disclosed herein may be used to downregulate the production or over production of one or more nesprin-2 that may lead to undesirable effects on the skin. The over production of nesprin-2 proteins may lead to excessive or abnormal skin growth resulting in undesirable effects such as skin diseases or disorders characterized by overgrowths of the skin, scleroderma, and/or wrinkles. Examples of such conditions may include, scars such as keloid scars and cutaneous effects of scleroderma such as sclerodactylia, acrosclerosis, and/or telangiectasis.

Further, the modulators of the current invention may impart anti-aging benefits as well by treating, ameliorating or preventing the weakening and/or compromised nucleoskeletal/cytoskeletal linkage associated with aging. The signs of aging are manifested in the wrinkles, folds, and pouches such as those found along the forehead; cheeks; around the eyes, and most prominently under the eyes; at the corners of the mouth; under the chin; or on the neck. As noted above, these undesirable features are due in part to the weakening of the nucleoskeletal/cytoskeletal/extracellular matrix. The method of the current invention may be used to rejuvenate, repair, and/or strengthen the skin and thereby prevent, forestall, ameliorate, and/or treat these undesirable signs of aging.

The composition will typically be applied to the skin one, two, or three times daily for as long as is necessary to achieve desired smoothing results. The treatment regimen may comprise daily application for at least one week, at least two weeks, at least four weeks, at least eight weeks, or at least twelve weeks. Chronic treatment regimens are also contemplated.

In a specific embodiment, the nesprin-2 modulator is provided in a pharmaceutically, physiologically, cosmetically, and dermatologically-acceptable vehicle, diluent, or carrier, where the composition is topically applied to an affected area of skin and left to remain on the affected area in an amount effective for improving the condition and aesthetic appearance of skin.

The method of the invention may be employed prophylactically to forestall aging including in patients that have not manifested signs of skin aging, most commonly individuals under 25 years of age. The method may also reverse or treat signs of aging once manifested as is common in patients over 25 years of age.

### Example 1

### Nesprin-2 mRNA Expression Decreases With Chronologic Age

Dermal fibroblasts were plated on 10 cm dishes. Cells were grown to subconfluence and were starved overnight in basal medium. The following day the medium was removed, the cells washed once with cold PBS. RNA was isolated from the cells using RNAqueous kit according to the manufacturer's instructions. Initial RNA concentration was determined by UV analysis. RNA integrity and concentration was verified by an Agilent Bioanalyzer 2100 using an RNA Nano Chip. cDNA was prepared using ABI High Capacity cDNA Reverse Transcription Kit with manufacturer's procedures. Taqman primer/probe set was obtained from ABI (SYNE2, Spectrin repeat containing nuclear envelope 2: Hs00794881_ml) Gene expression analysis was performed using the isolated RNA and an ABI 7900 HT RT-PCR machine.

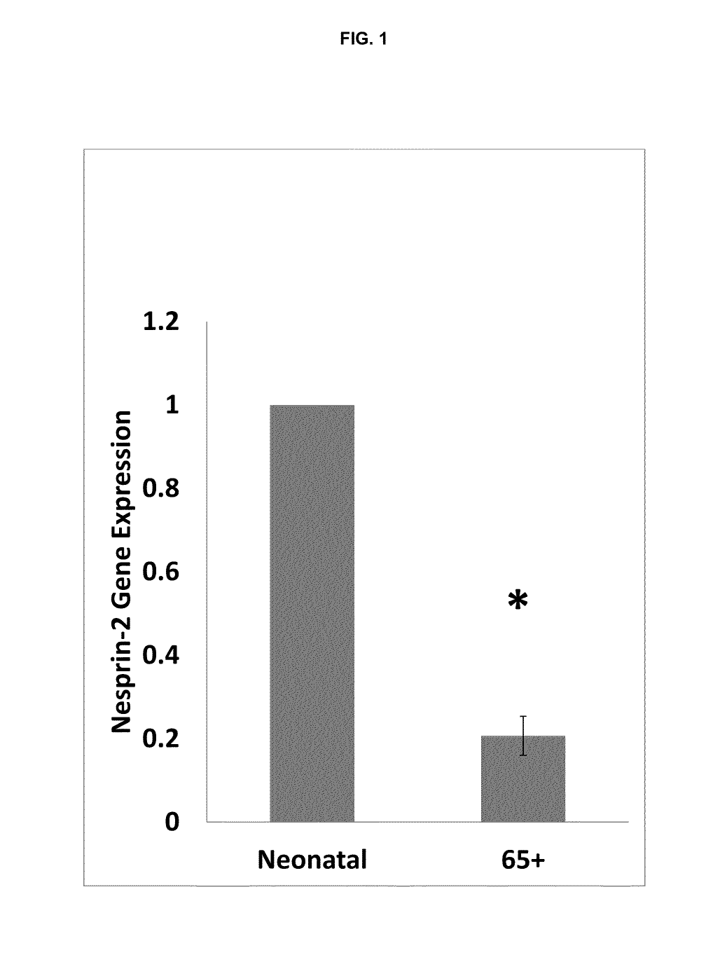

Five cell lines were used from each age group. The results are summarized in FIG. 1 and demonstrate that Nesprin-2 mRNA expression decreases with chronologic age.

### Example 2

### Nesprin-2 Protein Expression Decreases With Photoaging And With Chronologic Aging

30 healthy female Caucasian subjects in two age groups (Young 18-25 yr-old, n=15; aged 65-80 yr-old, n=15), with skin type II or III were recruited for biopsy study. A 4 mm punch biopsy was obtained from middle dorsal forearm and middle ventral upper arm from each subject. Tissue samples were fixed in 10% buffered formalin, embedded in paraffin, sectioned for 5 um thickness, and stained for Nesprin2 after re-hydration.

In all 30 subjects, photo-exposed skin has less Nesprin2 staining compared to photo-protected skin.

In photo-exposed skin, samples from all 15 aged subjects have less Nesprin-2 staining comparing to samples from young subjects.

### Example 3

### Assay for Upregulation of Nesprin-2

A variety of botanical extract and synthetic compounds were tested for the ability to up-regulate Nesprin-2 (SYNE2), Gene Accession Number—NM_{—}182914.2. Normal human dermal fibroblasts or keratinocytes were cultured in 96-well tissue culture treated plates, containing appropriate culture medium. Stock solutions of actives were made in an appropriate vehicle (water/Ethanol/DMSO). Cells were treated with test material or respective vehicle control diluted in growth medium for 24 hours in a humidified 37° C. incubator with 10% CO₂. The concentrations of each extract are provided based on the dry weight of the given plant extract, by which is meant the weight of the extract after volatile extraction solvents have been removed. After incubation, growth medium from each plate was removed and 100 μL of lysis buffer was added to the wells and placed in 37° C. incubator with 10% CO₂ for 30 minutes. At the end of incubation, the cells are collected in freezer plates and placed in −80° C. freezer, until analysis. Changes in mRNA for after treatment were analysed using Panomics Quantigene multiplex assay that employs a branched DNA technology, following manufacturer's instructions (Affymetrix, CA). Percent increase (up-regulation) in mRNA for the Nesprin-2 was calculated by comparing the test results to the control. The percent up-regulation is converted to a scaled score as shown in TABLE 1 below.

TABLE 1

| Active Ingredients for Nesprin2 | | |
|---|---|---|
| Stimulation | Nesprin-2 | Cell Type |
| Medemia noblis | ++++ (0.0001%) | Fibroblasts |
| D-Desthiobiotin | ++++ (0.001%, | Fibroblasts |
| | 0.0001%) | |
| Ixora chinensis | ++++ (0.001%) | Keratinocytes |
| Tiliacora triandra | + (0.01%) | Keratinocytes |
| Operculina turpethum | ++++ (0.1%, | Keratinocytes |
| | 0.01%) | |
| Callistephus chinensis | ++++ (0.1%), +++ | Keratinocytes |
| | (0.01%) | |
| Hoya carnosa | ++++ (0.1%) | Keratinocytes |
| Serrissa japonica | +++ (0.1%), ++++ | Keratinocytes |
| | (0.01%) | |
| Archidendron clyperia | ++++ (0.01%) | Keratinocytes |
| Justicia ventricosa | ++++ (0.1%) | Keratinocytes |

Key:
20-39 (+);
40-59 (++);
60-79 (+++);
>80 (++++)

### Example 4

### Exemplary HPLC protocol

Extracts were generally characterized by high performance liquid chromatography. A sample size of approximately 5 mg/mL was dispersed in 25/75 MeOH/H₂O and sonicated. The characterization was performed on a Zorbax SBC-18 column (7.5 cm×4.6 mm, 3.5 um particle size) and detection was achieved using diode array UV absorbance, 260 nm 300 nm and 360 nm, with lines on FIG. 1 depicted in ascending order and 260 nm on bottom. In one embodiment, In one embodiment, the extracted composition of a compound, in substantial isolation, exhibits an HPLC profile substantially similar to that depicted herein.

Operating conditions were flow rate 1.5 ml/min; temperature, 40° C.; sample injection volume, 20 μL, and time of run, 19 minutes. The mobile phase gradient used was as follows:

TABLE 2
Mobile Phase Gradient

| Time | Phase |
|---|---|
| 0 Minutes: | 15% Methanol(Solvent B)/85% Water with 1% acetic |
| | acid (Solvent A) |
| 10 Minutes: | 95% Methanol/5% Water with 1% Acetic acid. |
| 15 Minutes: | 15% Methanol/85% Water with1% Acetic acid. |
| 15.01 Minutes | 95% Methanol/5% Water with1% Acetic acid. |
| 19 Minutes: | 15% Methanol/85% Water with1% Acetic acid |

### Example 5

### Preparation of Extracts

A. Preparation of Tiliacora triandra Extract.

Preparation of Tiliacora triandra extract is generally described in U.S. patent application Ser. No. 12/345,707, filed on Dec. 30, 2008; Ser. No. 13/158,947, filed on Jun. 30, 2010; and Ser. No. 12/827,001, filed on Jun. 30, 2010, the entirety of which is incorporated by reference for all purposes. (Exhibits 4-6). Tiliacora triandra may be extracted from natural raw materials using methods of aqueous organic solvent extraction as is well known in the art. Two such extraction processes are set forth below.

1. Extraction of Tiliacora Triandra by Ethanol

An extract was obtained by extracting the vine of the Tiliacora triandra plant using an ethanol extraction scheme. Briefly, the vines of Tiliacora triandra Diels were first manually ground into small particles resulting in a powder of about 250 grams per flask (2 flasks). The ground powder was then extracted with 80% ethanol (2×2,000 ml per flask). After filtering and vacuum evaporation, the total concentrated extract was lyophilized resulting in an ethanolic extract of 50 grams. Tannins were removed resulting in an ethanolic extract of Tiliacora triandra of 46.04 grams.

2. Extraction of Tiliacora Triandra by Hexane

An extract was obtained by extracting the vine of the Tiliacora triandra plant using a hexane extraction scheme. Briefly, the vines of the Tiliacora triandra were first manually ground into small particles resulting in a powder of about 250 grams per flask (2 flasks). The ground powder was then extracted with 100% hexane (2×2,000 ml per flask). After filtering and vacuum evaporation, the total concentrated extract was dried by hot air oven at 40° C. resulting in an hexanolic extract of Tiliacora triandra of 0.61 grams.

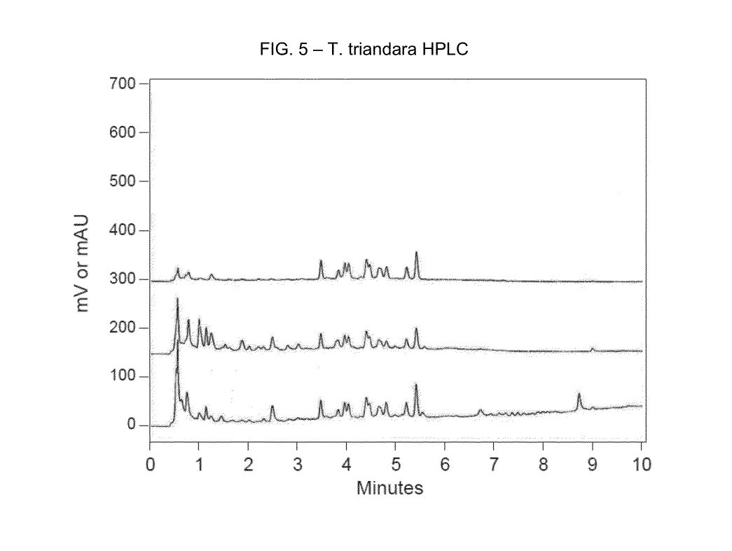

A HPLC trace of a representative Tiliacora triandra extract is found at FIG. 5.

B. Preparation of Medemia nobilis Extract.

Preparation of Medemia nobilis extract is generally described in U.S. patent application Ser. Nos. 13/305,779 and 13/216,626, filed on Dec. 30, 2010 and Aug. 24, 2011, respectively, the entirety of each of which is hereby incorporated by reference in their entirety for all purposes. Medemia nobilis leaves and stems are extracted with water/ethanol, and filtered to generate a Medemia nobilis raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Medemia nobilis extract, which may then be resuspended for further use (in one embodiment, as an aqueous resuspension).

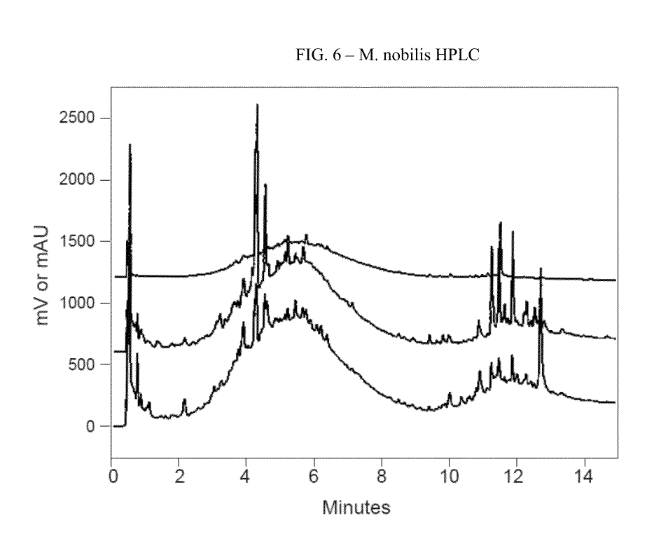

A HPLC trace of a representative Medemia nobilis extract is found at FIG. 6.

C. Preparation of Ixora chinensis Extract

Preparation of Ixora chinensis extract is generally described in U.S. patent application Ser. No. 13/158,947, filed on Jun. 30, 2010, the entirety of which is incorporated by reference for all purposes and U.S. patent application Ser. No. 13/324,150, filed on Dec. 13, 2011, the entirety of which is incorporated by reference for all purposes. An extract is obtained by extracting the dry chopped plant of Ixora chinensis Lamk. using an ethanol extraction followed by a further extraction with hexane. Briefly, the chopped plant of Ixora chinensis is first manually ground into small particles resulting in a powder of about 250 grams. The ground powder is then extracted with 50% ethanol. After filtering and vacuum evaporation, the total concentrated extract is diluted with water, centrifuged and filtered. The liquid is then thrice extracted with hexane, the hexane upper layer being discarded and the aqueous layer being lyophilized resulting in an extract of about 90 grams.

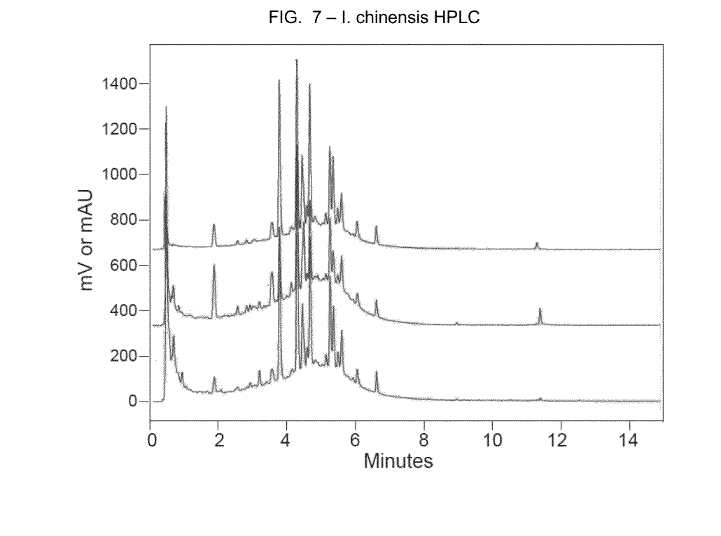

A HPLC trace of a representative Ixora chinensis extract is found at FIG. 7.

D. Characterization of D-Desthiobiotin

Preparation of D-desthiobiotin is generally described in U.S. patent application Ser. No. 12/747,364, filed on Dec. 21, 2007, the entirety of which is incorporated by reference for all purposes. In a particular embodiment, the compound is desthiobiotin which has the structure:

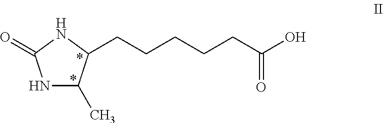

This compound may be present as the (R,R), (R,S), (S,R), or (S,S) stereoisomer, or as a mixture of two or more of these stereoisomers. Formula II includes the compounds L-Desthiobiotin, D-Desthiobiotin, and DL-desthiobiotin. In one embodiment, the stereocenter to which the methyl group is attached is in the S configuration and the other stereocenter is in the R configuration, as shown below:

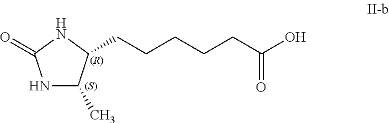

The compound of formula II is D-Desthiobiotin (CAS Registry No. 533-48-2), and is also known as (+)-Dethiobiotin or d-Dethiobiotin.

The compounds described herein can be present in the form of topically acceptable salts or complexes (e.g., non-toxic and/or non-irritating) that retain the biological activity of the compound. The salts may be either inorganic or organic acid or base addition salts. Suitable acid salts include but are not limited to acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfate, glucoheptonate, glycerophosphate, hemisulfate, heptonate, hexanoate, fumarate, hydrochloride, hydrobromide, hydroiodide, 2-hydroxy-ethanesulfate, lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, tosylate, and undecanoate. Special mention may be made of hydrochloride salts. Base addition salts include those formed with metal cations such as zinc, calcium, bismuth, barium, magnesium, manganese, lithium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine, etc. Any nitrogen-containing groups, can be quaternized with lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides such as benzyl and phenethyl bromides, to name a few.

Suitable prodrugs of desthiobiotin and its analogs (e.g., compounds of formula I) include those having the structures of formulas IIIa or IIIb:

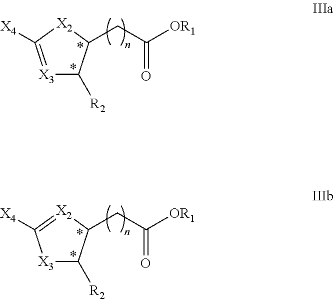

In structure IIIa, X₃ is N and X₄ is OR₁ or NR^{N}R₁; where R^{N}, R₁, R₂, and X₂ are defined as above. In structure IIIb, X₂ is N and X₄ is OR₁ or NR^{N}R₁; where R^{N}, R₁, R₂, and X₃ are defined as above. The prodrugs hydrolyze in vivo to the compounds of formula I.

E. Preparation of Justicia ventricosa Extract

Preparation of Justicia ventricosa extract is generally described in U.S. patent application Ser. No. 13/216,626, filed on Aug. 24, 2011, the entirety of which is incorporated by reference for all purposes. The botanical materials may be gathered and pulverized. Subsequently, reflux extraction, using methods known to those of ordinary skill in the art, may be conducted using 8-10× the weight of the pulverized flowers of water at 100° C. This step may be repeated. The resulting extract may be filtered and condensed using methods known to those of ordinary skill in the art, after which the extract may undergo vacuum distillation at appropriate conditions known to those of ordinary skill in the art. Subsequently, the extract may be mixed with a suitable amount of dextrin and spray dried.

As noted in the remaining specification, modifications and adaptations of the above-noted extraction process are possible, particularly during a scale-up to larger volumes for production.

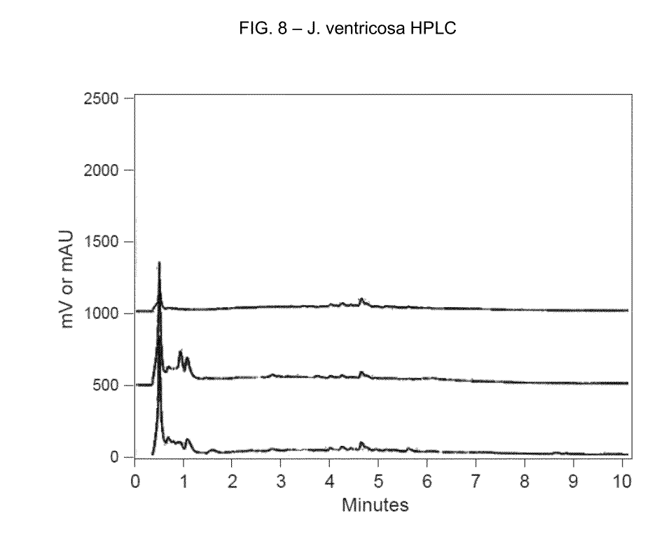

A HPLC trace of a representative Justicia ventricosa extract is found at FIG. 8.

F. Preparation of Archidendron clyperia Extract

Preparation of Archidendron clyperia extract is generally described in U.S. patent application Ser. No. 13/216,626, filed on Aug. 24, 2011, the entirety of which is incorporated by reference for all purposes. The botanical materials may be gathered and pulverized. Subsequently, reflux extraction, using methods known to those of ordinary skill in the art, may be conducted using 8-10× the weight of the pulverized flowers of water at 100° C. This step may be repeated. The resulting extract may be filtered and condensed using methods known to those of ordinary skill in the art, after which the extract may undergo vacuum distillation at appropriate conditions known to those of ordinary skill in the art. Subsequently, the extract may be mixed with a suitable amount of dextrin and spray dried.

As noted in the remaining specification, modifications and adaptations of the above-noted extraction process are possible, particularly during a scale-up to larger volumes for production.

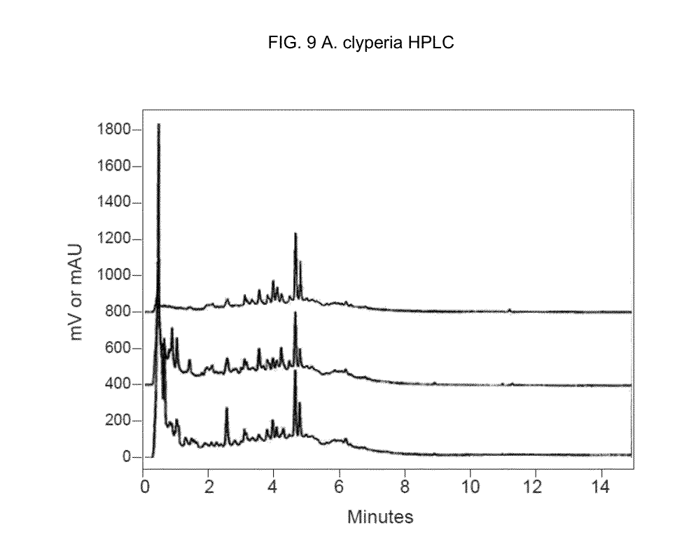

A HPLC trace of a representative Archidendron clyperia extract is found at FIG. 9.

G. Preparation of Serrisa japonica Extract

Preparation of Serrisa japonica extract is generally described in PCT Patent Application Serial No. PCT/US12/68858, the entirety of which is incorporated by reference herein for all purposes.

### Extraction Protocol

I. Extraction

250 g of dried and powdered material derived from the leaves and/or stems of Serissa japonica was percolated with 1000 ml EtOH/H₂0 (50:50, v/v) at room temperature for 24 hours. This percolation was repeated 2 times and then the EtOH/H₂0 extraction solution was concentrated under vacuum by rotary evaporator at 40-50° C. to 150 ml or end of distillation, whichever occurs first.

The concentrated solution was then diluted with pure water to 1500 ml of volume and sonicated for 20 minutes to generate an aqueous suspension. The suspension was left to stand at 4° C. for 12 h and then centrifuged. The supernatant was then transferred to a separation funnel where three separate extraction were done with 500 ml of hexane each. The hexane solvent was recycled, and the hexane extract was discarded.

Charcoal (10% by w. vs. total dry matter content) was then added to the aqueous phase yielded from the hexane extraction and stirred for 1 hour. The solution was then filtered and concentrated under vacuum at 40-50° C. to adjust the concentration of solution to 5% (w/v) of its dry matter. The adjusted solution was then passed through a Diaion HP-20 column (20 times of the dry weight,) and washed successively with:

H20: 2 times Diaion HP-20 column volume (100 g HP-20 is equal to 210 ml of column volume);

2) 20% aqueous EtOH: 2 times Diaion HP-20 column volume;

3) 50% aqueous EtOH: 2 times Diaion HP-20 column volume;

4) 95% aqueous EtOH: 3 times Diaion HP-20 column volume.

The elutents of washes 1-4 were concentrated, respectively, to dryness to obtain fractions 1-4.

As noted in the remaining specification, modifications and adaptations of this extraction process are possible, particularly during a scale-up to larger volumes for production.

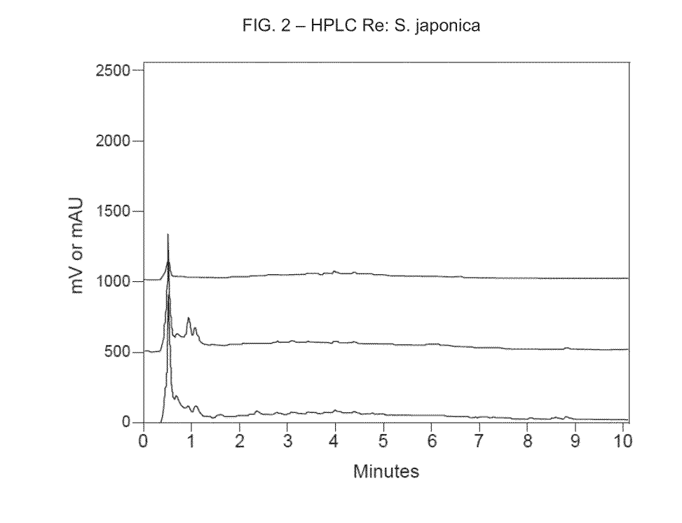

A HPLC trace of a representative Serissa japonica extract is found at FIG. 2.

H. Preparation of Callistephus chinensis Extract

Preparation of Callistephus chinensis extract is generally described in PCT Application Serial No. PCT/US12/68856, the entirety of which is incorporated by reference herein for all purposes.

250 g of chopped Callistephus chinensis leaves and/or stems were gathered an then dried in an electric oven at 60° C. for 2 or 3 days. The dried leaves and/or stems were then added to 1 L of 50% hydroethanol (EtOHIH₂0 50-50 v/v; 3×4 volume) in a container and extraction occurred by shaking the contaner at 150 rpm at 37° C. for 12 h. This extraction was repeated three times to achieve 3L of total extract. The total extract was then subjected to vacuum concentration using a rotary evaporator at a temperature of 40-50° C. until the volume was reduced to about 150 m. The concentrated fraction was then diluted with pure water to 1500 ml and was left to stand at 4° C. for 12 h (or more) and then centrifuged to remove any residue. The 1500 ml of the now clarified solution was treated, twice, with 750 ml of hexane in a separation funnel and the organic layer was discarded. The dry content of the remaining aqueous homogenous solution was confirmed by taking a 150 ml sample of the homogenous solution and lyophilizing the sample. The resulting powder was weighed and used to calculate the total amount of dry matter in the homogenous solution. The resulting powder was then redissolved in water (about 100-150 ml) and pooled with the homogenous solution. 10% by weight of charcoal was then added to the homogenous solution, about 17 g of charcoal for a solution of about 1500 ml. The solution was then stirred at 50° C. for 1 hour and then filtered on a filter paper (type, manufacturer) and the step was repeated. Next, the now clear solution is fractioned by liquid/liquid extraction with water saturated n-butanol. The saturated butanol was prepared by adding to butanol with the same volume of water in a separating funnel, after mixing the organic upper layer was collected and used for the liquid/liquid of clear solution. About 2250 ml of the water saturated butanol was prepared for the 1500 ml clear solution. The water saturated butanol was divided into three 750 ml portions and each was used to treat the clear solution three times in a separatory funnel. The resulting organic layer (butanolic extract) and aqueous layers from each run through the separatory funnel were collected and pooled separately. First, an equal volume of water was added to the pooled butanolic extract and the solution was concentrated in a rotary evaporator under vacuum. When the distillation stopped an equal volume of water was added and the concentration was repeated. The concentration was repeated a third time and the resulting solution was lyophilized (first purified extract). Second, the aqueous layer resulting from each run through the separatory funnel was concentrated by rotary evaporator under vacuum to remove butanol (azeotrope boiling point is less than 100° C., bath temperature 60° C.). When distillation stopped, the aqueous solution was lyphylized (second purified extract). The first and second extracts were then combined and weighed.

As noted in the remaining specification, modifications and adaptations of this extraction process are possible, particularly during a scale-up to larger volumes for production.

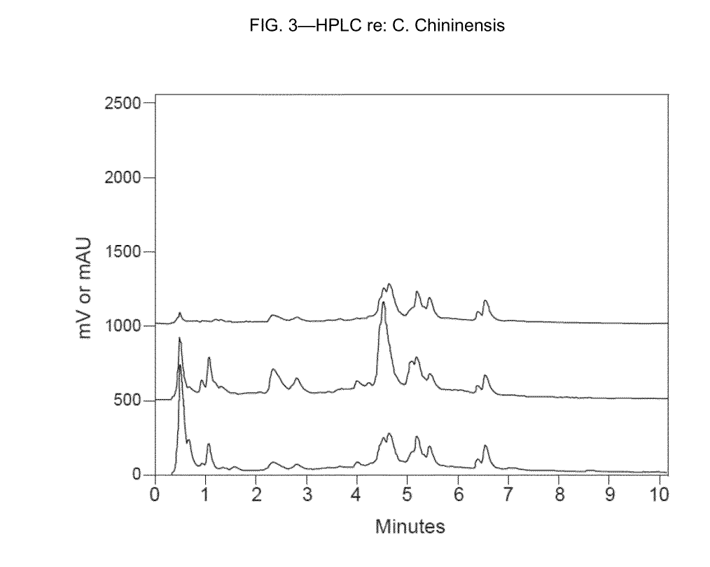

A HPLC trace of a representative Callistephus chinensis extract is found at FIG. 3.

I. Preparation of Hoya carnosa Extract

Preparation of Hoya carnosa extract is generally described in PCT Application Serial No. PCT/US12/68865, the entirety of which is incorporated by reference herein for all purposes

I. Preparation of Purified Plant Extract

250 g of dried and powdered material derived from the leaves and/or stems of Hoya carnosa was percolated with 1000 ml EtOH/H₂0 (50:50, v/v) at room temperature for 24 hours. This percolation was repeated 2 times and then the EtOH/H₂0 extraction solution was concentrated under vacuum by rotary evaporator at 40-50° C. to 150 ml or end of distillation, whichever occurs first.

The concentrated solution was then diluted with pure water to 1500 ml of volume and sonicated for 20 minutes to generate an aqueous suspension. The suspension was left to stand at 4° C. for 12 h and then centrifuged. The supernatant was then transferred to a separation funnel where three separate extraction were done with 500 ml of hexane each. The hexane solvent was recycled, and the hexane extract was discarded.

Charcoal (10% by w. vs. total dry matter content) was then added to the aqueous phase yielded from the hexane extraction and stirred for 1 hour. The solution was then filtered and concentrated under vacuum at 40-50° C. to adjust the concentration of solution to 5% (w/v) of its dry matter. The adjusted solution was then passed through a Diaion HP-20 column (20 times of the dry weight,) and washed successively with:

1) H20: 2 times Diaion HP-20 column volume (100 g HP-20 is equal to 210 ml of column volume);

2) 20% aqueous EtOH: 2 times Diaion HP-20 column volume;

3) 50% aqueous EtOH: 2 times Diaion HP-20 column volume;

4) 95% aqueous EtOH: 3 times Diaion HP-20 column volume.

The elutents of washes 1-4 were concentrated, respectively, to dryness to obtain fractions 1-4.

As noted in the remaining specification, modifications and adaptations of this extraction process are possible, particularly during a scale-up to larger volumes for production.

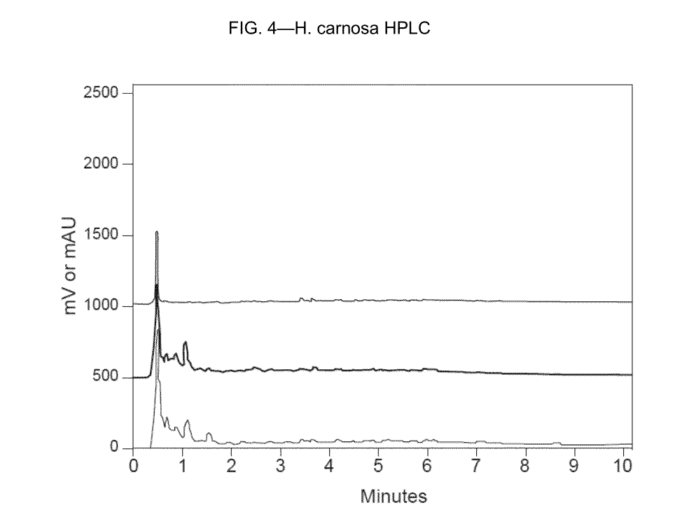

A HPLC trace of a representative Hoya carnosa extract is found at FIG. 4.

J. Preparation of Operculina turpethum Extract

Preparation of Operculina turpethum extract is generally described in U.S. patent application Ser. Nos. 13/305,779 and 13/216,626, filed on Dec. 30, 2010 and Aug. 24, 2011, respectively. Operculina turpethum is extracted with water/ethanol, and filtered to generate a Operculina turpethum raw extract. The extract is then concentrated to aqueous suspension, which is let stand overnight at 4 C. The concentrated aqueous suspension is then precipitated and filtered with solid fraction removed, yielding a filtered aqueous filtered solution. Butanol is then added to the filtered aqueous suspension, then a liquid/liquid extraction is performed with subsequent removal of the organic phase. The remaining aqueous phases is then concentrated, dried, and irradiated to yield a dried purified Operculina turpethum extract, which may then be resuspended for further use (in one embodiment, as an aqueous resuspension).

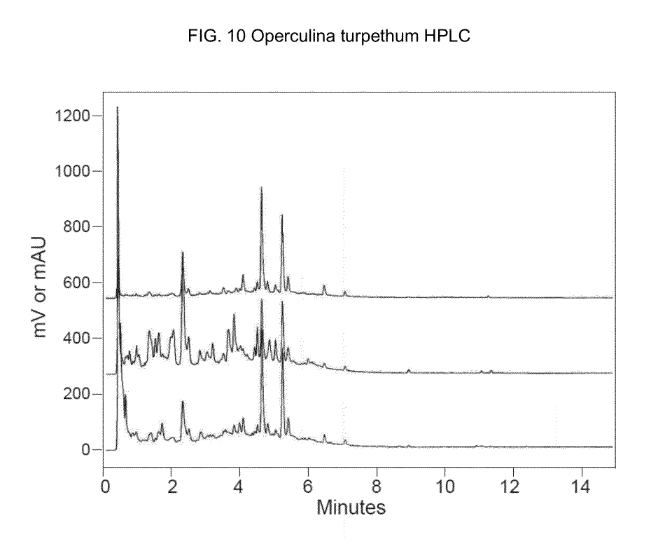

A HPLC trace of a representative Operculina turpethum extract is found at FIG. 10.

### Example 6

### A. Exemplary Anti-Cellulite Compositions

Cosmetic compositions comprising an nesprin-2 modulator for topical application to skin exhibiting or at risk of exhibiting impaired cytoskeleton and/or nuclear envelope integrity, loss of proper cell polarity and/or alignment, and disregulation of wound healing and/or skin regeneration, which may lead to cellulite are provided in Table 3.

TABLE 3
Sample Cosmetic Composition
Ingredient

Aesthetic modifier
Emollient
Emulsifier
Anti-inflammation agent
Chelater
Coolant
Elastin stimulator
Exfoliator
Fragrance
Humectant
Microcirculation enhancer
Neutralizer
Preservative
Sunscreen
Collagenase/elastinase inhibitor
Hawthorne (Crataeg. monog.) Fruit. Extract
Coffee Seed Extract
Soybean (Glycine soja) Extract
Celosia cristata Extract & Prunella vulgaris Extract
L-Carnitine Hydrochloride
Averrhoa carambola Leaf Extract
Nesprin-2 modulator
Demineralized water

### B. Exemplary Anti-Aging Facial Cosmetic Composition

Cosmetic compositions comprising a nesprin-2 modulator for topical application to areas of the face exhibiting or at risk of exhibiting signs of aging due to a reduction in the amount of nesprin-2 are provided in Table 4.

TABLE 4
Sample Anti-aging Facial Cosmetic Composition
Ingredient

Aesthetic modifier
Emollient
Emulsifier
Anti-inflammation agent
Chelater
Coolant
Elastin stimulator
Exfoliator
Fragrance
Humectant
Microcirculation enhancer
Neutralizer
Preservative
Sunscreen
Collagenase/elastinase inhibitor
Phytol
Antioxidant
Fennel Extract
Carrot extract
Pomegranate extract
Thiodipropionic acid (TDPA)
Green tea polyphenol
L-4 Thiazolylanine
Nesprin-2 modulator
Demineralized water

### C. Exemplary Skin Lightening Compositions

Cosmetic compositions comprising a nesprin-2 modulator for topical application to skin exhibiting signs of hyperpigmentation are provided in Table 5.

TABLE 5
Sample Skin Lightening Compositions
Description

Demineralized Water
Carbopol 934
Acrylates/C10-30 Alkyl Acrylate Crosspolymer
Acrylates/C10-30 Alkyl Acrylate Crosspolymer
Xanthan Gum
Disodium EDTA - Tech Grade
Methylparaben
Alcohol SD40B
Alcohol Mixture (3210&1901 92.52-7.48)
Alcohol Mixture (3215&1901 92.52-7.48)
Phenoxyethanol-98% MIN (*RI*)
Butylene Glycol
Pentylene Glycol (*RI*)
Ethoxydiglycol
ISODODECANE
Dilauryl Thiodipropionate
Tetrahexyldecyl Ascorbate
Ascorbyl Glucoside
Glycyrrhizinate - Dipotassium Unp.
Silica Shells
Sodium Hydroxide Solution 50%
Silicone Fluid SF-96-5
PEG-40 Stearate
Steareth-2
Saxifraga Sarmentosa/Grape Extract
Saccharomyces/Zinc ferment
Yeast Extract
Kudzu (Pueraria Lobata) Symbiosome extract
Soybean (Gly. Soja) Extract
Carrot (Daucus Carota Sativa) Root Extract
Phytol
Dimethicone/Dimethicone Crosspolymer
Thiodipropionic Acid
Nesprin-2 modulator

These compositions are believed to be effective to treat, reverse, ameliorate and/or prevent signs of skin aging, specifically, the compositions are believed to reduce the appearance of skin irregularities, such as impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging on the body and under-eye bags on the face. The compositions of Tables 3-5 are applied to skin in need of treatment, by which is meant skin in need of an anti-impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging benefit, and in particular skin exhibiting irregularities due to a downregulation of nesprin-2 expression such as impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging on the abdomen, thighs, buttocks or limbs. The compositions of Tables 3-5 are applied to the facial skin in need of treatment, by which is meant skin in need of an anti-aging benefits. These cosmetic compositions may be applied directly to the affected areas of skin, i.e., skin exhibiting the impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging or periorbital bulging.

The cosmetic compositions are applied to the skin exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging and/or eye bag one, two or three times daily for as long as is necessary to achieve desired results, which treatment regimen may comprise daily application for at least one week, at least two weeks, at least four weeks, at least eight weeks, or at least twelve weeks. Alternatively, the exemplary cosmetic compositions may be used in chronic treatment of the skin exhibiting impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging and/or eye bags in need thereof.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We hereby claim:

1. A method for improving the appearance of skin affected by a skin irregularity comprising topically applying thereto an effective amount of at least one modulator of nesprin-2, in a cosmetically acceptable vehicle for a time sufficient to achieve an improvement in the appearance of said skin.

2. The method according to claim 1, wherein the modulator upregulates an nesprin-2 protein.

3. The method according to claim 1, wherein the modulator downregulates an a nesprin-2 protein.

4. The method according to claim 1, wherein said skin irregularity is impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging.

5. The method according to claim 1, wherein said achievement in the appearance of the skin is selected from the group consisting of:
(a) reduction in the appearance of skin affected by impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging;
(b) reduction in pitting appearance of skin affected by impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging upon squeezing;
(c) reduction in the extent of skin affected by impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging;
(d) prevention or delay in the recurrence of impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging; or
(e) improvement in collagen disposition.

6. The method of claim 5, wherein said skin irregularity is sagging of the facial skin.

7. The method of claim 1, wherein the modulator comprises an extract of Justicia ventricosa, an extract of Tiliacora triandara, an extract of Ixora chinensis, an extract of Operculina turpethum, D-desthiobiotin or a derivative thereof, an extract of Archidendron clyperia, an extract of Medemia nobilis, an extract of Serrisa japonica, an extract of Callistephus chinensis, an extract of Hoya carnosa, or a combination thereof.

8. The method of claim 7, wherein the modulator is not an extract of Justicia ventricosa, Tiliacora triandra, Ixora chininensis, Operculina turpethum, Archidendron clyperia, Medemia nobilis,
or a combination thereof.

9. The method of claim 7, wherein the modulator is not D-desthiobiotin or a derivative thereof.

10. The method of claim 1, wherein the modulator is in combination with at least one other anti-lipid agent.

11. The method of claim 1, wherein the at least one anti-impaired cytoskeleton and/or nuclear envelope integrity, loss of proper cell polarity and/or alignment, and disregulation of wound healing and/or skin regeneration, which may lead to lines/wrinkles, sagging, and other signs of aging and/or photoaging agent is selected from the group consisting of a phophodiesterase inhibitor, an adenylate cyclase activator, a lipolysis stimulator, a beta-adrenergic receptor agonist, an alpha-2-adreneric receptor antagonist, perilla oil, carnitine, creatine, or combination thereof.

12. The method according to claim 1, wherein said modulator is in combination with at least one collagen and/or elastin stimulator.

13. The method of claim 1, wherein an effective amount of the modulator is about 0.001% to about 25% by weight of a composition.

14. A method for screening active agents useful for improving the aesthetic appearance of skin comprising assaying candidate substances for ability to modify nesprin-2 expression.

15. A method of treating the skin comprising topically applying to an area of the skin in need thereof an effective amount of an active agent that modulates nesprin-2expression, wherein the ability of said active agent to modulate nesprin-2expression has been determined by an assay which measures the level of mRNA encoding nesprin-2 in a cell that has been contacted with said active agent.
